(19) United States
(12) Patent Application Publication
(10) Pub. No.: US 2025/0268661 A1
(43) Pub. Date: Aug. 28, 2025

(54) APPARATUS AND METHODS FOR PERFORMING A MEDICAL PROCEDURE

(71) Applicant: SurgiAI LTD, Olesh (IL)

(72) Inventors: Nissan Elimelech, Beerotaim (IL); Noam Racheli, Olesh (IL)

(21) Appl. No.: 19/204,871

(22) Filed: May 12, 2025

Related U.S. Application Data
(63) Continuation of application No. PCT/IB2023/061590, filed on Nov. 16, 2023.
(60) Provisional application No. 63/425,725, filed on Nov. 16, 2022.
(60) Provisional application No. 63/472,006, filed on Jun. 9, 2023.

(51) Int. Cl. A61B 34/20; A61B 34/10; A61B 90/00; G06T 3/18; G06T 7/00; G06T 7/11; G06T 7/30; G06T 7/50; G06T 17/00

(52) U.S. Cl. CPC A61B 34/20; G06T 3/18; G06T 7/0012; G06T 7/11; G06T 7/30; G06T 7/50; G06T 17/00; A61B 2034/107; A61B 2034/2055; A61B 2034/2065; A61B 2090/367; A61B 2090/373; G06T 2207/10048; G06T 2207/30004; G06T 2210/41

(57) ABSTRACT

Apparatus and methods are described for use during a surgical procedure that is performed on a portion of a body of a patient using a surgical instrument. A computer processor segments the portion of the patient's body within intraoperative images and performs 3D reconstruction of the portion of the patient's body based on at least some of the intraoperative images. The computer processor coregisters the portion of the patient's body to a common coordinate system with the portion of the patient's body as it appears within the preoperative imaging data using a non-rigid coregistration algorithm and drives an output device to display an output, based upon the coregistering. Other applications are also described.

### CROSS-REFERENCES TO RELATED APPLICATIONS

The present application is a continuation of International Application No. PCT/IB2023/061590 to Elimelech, filed Nov. 16, 2023, entitled “Apparatus and method for performing a medical procedure,” which claims priority from:
U.S. Provisional Patent Application No. 63/425,725 to Elimelech, filed Nov. 16, 2022, entitled “Apparatus and method for performing a medical procedure,” and
U.S. Provisional Patent Application No. 63/472,006 to Elimelech, filed Jun. 9, 2023, entitled “Apparatus and method for performing a medical procedure.”

The above-referenced applications are incorporated herein by reference.

### FIELD OF EMBODIMENTS OF THE INVENTION

The present invention relates to methods and apparatus for use in medical procedures, and particularly surgical navigation apparatus and methods.

### BACKGROUND

Surgical navigation techniques are used in several different types of medical procedures, such as neurosurgery, spinal surgery, orthopedic surgery, and pulmonary procedures. Such techniques allow physicians to observe the current location of a surgical instrument with respect to preoperative imaging data. Typically, the preoperative imaging data includes CT and/or MRI images. In some cases, pre-planning of the surgery is performed with reference to the preoperative imaging data and the navigation allows the physician to observe the current location of the instrument with respect to a pre-planned trajectory and/or with respect to a pre-planned target, such as a lesion, a tumor, etc.

In order to perform the surgical navigation, the patient's anatomy is coregistered to the preoperative imaging data, such that corresponding points in the patient's anatomy and the preoperative imaging data are aligned with each other within a common coordinate system. Typically fiducial markers are placed on the patient's body as well as on the surgical instrument, such that the location of the surgical instrument with respect to the patient's anatomy may be derived. By virtue of the coregistration of the patient's anatomy to the preoperative imaging data, the location of the surgical instrument with respect to the preoperative imaging data and/or with respect to the preoperative planning is thereby derived. Depending on the type of surgical navigation system that is employed the fiducial markers may be electromagnetic coils, or optical markers, e.g., reflective markers (and in some cases, reflective infrared markers) and/or radiopaque markers.

### SUMMARY OF EMBODIMENTS

In accordance with some applications of the present invention, surgical navigation is applied to surgical procedures that are performed on non-rigid tissue and/or on tissue that is prone to undergo movement, deformation, and/or resection (for example, in the case of bone that is cut) either during the procedure and/or between a presurgical image-acquisition stage and the surgery itself. Typically, prior to the procedure being performed, preoperative imaging data are acquired. For some applications, preoperative planning is performed with respect to the preoperative imaging data. For example, the trajectory of a surgical instrument through the patient's anatomy may be pre-planned using the preoperative imaging data. Alternatively or additionally, a target tissue, such as a lesion or a tumor may be located within the preoperative imaging data. For some applications, the preoperative planning includes planning the delivery and/or the deployment of an implant, for example, the implantation of an electrode in the brain, and/or the implantation of a cage (or other implant) in the spine.

As described in the Background section, typically prior art surgical navigation techniques involve coregistering the patient's anatomy to the preoperative imaging data, such that corresponding points in the patient's anatomy and the preoperative imaging data are aligned with each other, within a common coordinate system. Typically, fiducial markers are placed on the patient's body as well as on the surgical instrument, such that the location of the surgical instrument with respect to the patient's anatomy is derived. By virtue of the coregistration of the patient's anatomy to the preoperative imaging data, the location of the surgical instrument with respect to the preoperative imaging data and/or with respect to the preoperative planning is thereby derived. Typically, in such cases, the fixation of fiducial markers on the patient must be rigid and the fiducial markers are placed in fixed position relative to the anatomy that is to be navigated. Furthermore, if the anatomy moves during surgery, or the fiducial markers move with respect to the patient, the coregistration procedure must be performed again. Moreover, if the anatomy that is to be treated changes during surgery (e.g., due to tissue being cut, bones being broken, etc.), navigation cannot be used since preoperative imaging is no longer an accurate representation of the current anatomy.

Due to the aforementioned limitations, soft-tissue organs and/or tissue that is modified during surgery (e.g., due to tissue being cut, bones being broken, etc.) cannot be navigated with high accuracy using the above-described techniques. This is because such techniques rely upon the fiducial markers being maintained in rigidly-fixed positions with respect to the anatomy that is to be navigated, whereas soft tissue undergoes movement between the acquisition of the preoperative imaging date and the surgical procedure, and additionally undergoes movement during the surgical procedure (both as a result of natural movement as well as movement that is brought about by the interaction between the surgical instruments and the soft tissue). Therefore, in practice, surgical navigation is typically only performed on rigid anatomy such as bones, spine, and cars nose and throat (ENT). (In addition, surgical navigation is performed with respect to the lungs by using the network of airways as navigational guides.) In some cases, surgical navigation is performed in conjunction with brain surgery, based on the brain being encapsulated within the skull and therefore being held in a relatively fixed position. However, the brain sometimes move inside the skull when the skull is opened (in a phenomenon that is known as “brain shift”), and/or during surgery as a result of the surgery. Therefore, surgical navigation in brain surgery suffers from certain drawbacks. As described hereinabove, even in surgery that is performed with respect to rigid tissue, such as bones, the tissue often undergoes movement between the acquisition of the preoperative imaging date and the surgical procedure, and/or undergoes movement during the surgical procedure, e.g., as a result of bones being broken and/or moved, such that the coregistration becomes inaccurate.

Some applications of the present disclosure are directed toward overcoming the above-described limitations, such that surgical navigation is performed accurately with respect to soft tissue (e.g., organs such as the liver, spleen, or kidneys) and/or with respect to tissue that undergoes movement between the acquisition of the preoperative imaging date and the surgical procedure and/or undergoes movement during the surgical procedure (such as the brain). For example, some applications of the present disclosure are applied to operating upon vessels within the brain.

For some applications, the operating room includes an imaging system, such as a digital camera (and typically, a stereoscopic high-resolution camera). For some applications, the imaging system includes one or more red-green-blue (“RGB”) cameras, e.g., a pair of lenses and corresponding sensors arranged to provided stereoscopic vision. Alternatively or additionally, the imaging system includes one or more near-infrared (“NIR”) cameras, e.g., a pair of lenses and corresponding sensors arranged to provided stereoscopic vision. For some applications, the imaging system includes a hyperspectral camera.

For some applications, in an initial intraoperative step, the imaging system acquires a series of images at respective focal lengths. A computer processor identifies the region of interest within the series of images and thereby sets the focal length to be used by the imaging system for the further imaging steps that are to be performed by the imaging system. Typically, subsequent to the region of interest having been identified and the focal length derived, the imaging system acquires images of the surgical region of interest. For some applications, the computer processor identifies a portion of interest within the images of the surgical region of interest (e.g., an organ of interest, such as the kidney or liver, or one or more structures within an organ that are of interest, e.g., a given vessel or set of vessels within the brain) using an object-detection algorithm. For some applications, the computer processor runs an algorithm that has been pre-trained to identify the portion of the body. For example, the computer processor may run an algorithm that has pre-trained using machine-learning techniques, for example, a guided machine-learning algorithm, such as a convolutional neural network algorithm, using multiple real images of surgery with annotation of selected organs and structures. For some applications, the computer processor identifies the objects using a “You-Only-Look-Once” (“YOLO”) algorithm, a Single-Shot Detector (“SSD”) algorithm, and/or a Region-based Convolutional Neural Network (“R-CNN”) algorithm. In a subsequent step, the computer processor performs segmentation of the identified organ and/or structures. For some applications, the computer processor performs instance segmentation of the identified organ and structures using a YOLO algorithm, e.g., an INSTA-YOLO algorithm. Alternatively or additionally, a different type of segmentation algorithm (e.g., an SSD algorithm, and/or an R-CNN algorithm) is used.

Subsequent to the identified organ and structures being segmented, 3D reconstruction (mapping) of only the segmented organs or structures is performed. For some applications, the 3D reconstruction is performed by directing light toward the organ and/or structures of interest and detecting light reflected from the organ and/or structures using stereo vision and calculating the disparity between two sensors, e.g., the two sensors of a stereoscopic RGB camera, and/or the two sensors of a stereoscopic infrared camera. For some applications, light is directed toward the organ and/or structures of interest by a laser light source (e.g., a random structure laser light source) creating a pattern of laser light on the organ and/or structures. Typically, the laser light is visible light (which is configured to be captured by RGB cameras) and/or NIR laser light (configured to be captured by NIR cameras).

Subsequent to the 3D reconstruction of the organ and/or structures of interest, the organ and/or structures of interest are coregistered to the preoperative imaging data. The coregistration is typically performed using a coregistration algorithm that is applicable to non-rigid bodies. For some applications, the coregistration is performed using surface-matching registration method of non-rigid bodies. For some applications, the coregistration includes a step of deforming the preoperative imaging data to match current position and shape of the organ and/or structure of interest. For example, the preoperative imaging data (e.g., CT and/or MRI imaging data) of an organ may include data relating to the shape of a soft tissue organ which is different than the intraoperative shape of the organ in surgery. In addition, during the procedure, the shape of the organ may undergo changes (e.g., due to natural movement, due to movement of the organ by the surgical instruments, and/or due to the organ being cut). Typically, in such cases, the coregistration includes a step of deforming the preoperative imaging data to match the current position and shape of the organ and/or structure of interest. Typically, the computer processor determines how to deform the preoperative imaging data by (a) performing surface-matching registration to determine how to deform the surface of the organ within the preoperative imaging data, and then (b) modeling how the change in shape of the surface of the organ affects the shape of internal portions of the organ, based on mechanical models of behavior of the tissue that is present within the organ. For example, internal portions of the liver and brain will deform less than those of the bowels, while nerves will in some cases deform the most. Typically, by knowing the relationship between the surface of a given organ and the internal portions of the organ, the computer processor is able to accurately model how to deform the whole organ based upon the surface-matching registration.

For some applications, the imaging system includes a hyperspectral camera, and imaging data acquired using the hyperspectral camera are used to perform the coregistration of intraoperative imaging data with preoperative imaging data. For example, spectral imaging data that are indicative of a given tissue type may be used to perform the coregistration.

It is noted that, as described in the above paragraph, typically in cases in which an organ or a portion thereof is cut, then as part of the coregistration step, the preoperative imaging data is modified to create an accurate representation of the cut organ (typically, by removing parts of the preoperative imaging data corresponding to the part that has been cut). Typically, this increases the accuracy of the coregistration.

Typically once the coregistration has been performed, the physician navigates surgical instruments through the patient's anatomy, using the preoperative imaging data and/or preoperative planning to navigate.

There is therefore provided, in accordance with some applications of the present invention, apparatus for use during a surgical procedure that is performed on a portion of a body of a patient using a surgical instrument, an imaging system, an output device, fiducial markers placed upon the patient's body, and a surgical navigation system that is configured to coregister anatomy of the patient with the preoperative imaging data of the portion of the patient's body such that the patient's anatomy and the preoperative imaging data are registered with each other within a navigation system common coordinate system, by identifying the fiducial markers within images of the patient's body, the apparatus including:
at least one computer processor configured:
   to receive preoperative imaging data of the portion of the patient's body and the surgical instrument;
   segment the preoperative imaging data of the portion of the subject's body into substructures; and
   during the surgical procedure:
      to coregister the segmented substructure to the patient's body, such that the patient's body and the segmented substructures within the preoperative imaging data are registered within a navigation system common coordinate system;
      to coregister images acquired by the imaging system within the navigation system common coordinate system;
      to receive intraoperative images of the portion of the patient's body from the imaging system;
      to identify the portion of the patient's body within the intraoperative images;
      to segment the portion of the patient's body within the intraoperative images;
      to perform 3D reconstruction of the portion of the patient's body based on at least some of the intraoperative images;
      to coregister the portion of the patient's body to the preoperative imaging data within the navigation system common coordinate system, the coregistering including updating the preoperative imaging data by moving at least one of the segmented substructures relative to others of the segmented substructures; and
      drive the output device to display a current location of the surgical instrument with respect to the preoperative imaging data, based upon the coregistering.

In some embodiments, the computer processor is configured to segment the preoperative imaging data of the portion of the subject's body into substructures that are semi-rigid.

In some embodiments, the computer processor is configured to drive the output device to display the current location of the surgical instrument with respect to the preoperative imaging data, based upon the coregistering, without requiring use of instrument fiducial markers disposed on the surgical instrument.

In some embodiments, the surgical instrument includes instrument fiducial markers and the computer processor is configured to drive the output device to display the current location of the surgical instrument with respect to the preoperative imaging data at least partially by identifying the instrument fiducial markers within the intraoperative images.

In some embodiments, in response to detecting that the portion of the patient's body has undergone a change in shape since the preoperative imaging data were acquired, the computer processor is configured to update a shape of the preoperative imaging data to reflect the change in shape that the portion of the patient's body has undergone.

In some embodiments, the fiducial markers include fiducial markers that are visible within images acquired by the imaging system, and the computer processor is configured to coregister images acquired by the imaging system within the navigation system common coordinate system by identifying the fiducial markers within images acquired by the imaging system.

In some embodiments, the apparatus further includes markers coupled to the imaging system, and the computer processor is configured to coregister images acquired by the imaging system within the navigation system common coordinate system by tracking the markers that are coupled to the imaging system.

In some embodiments, the apparatus further includes the imaging system, the apparatus is configured for use with a surgical lighting system that includes a handle and at least a portion of the imaging system is disposed on a cover that is configured to be placed over the handle.

In some embodiments, the apparatus further includes the imaging system, the imaging system includes one or more infrared cameras that are configured to acquire images of the veins within the portion of the patient's body.

In some embodiments, the apparatus further includes the imaging system, the imaging system includes one or more depth cameras.

In some embodiments, the computer processor is configured, in real time with respect to the acquisition of the intraoperative images:
to identify the portion of the patient's body within the intraoperative images;
to segment the portion of the patient's body within the intraoperative images;
to perform 3D reconstruction of the portion of the patient's body based on at least some of the intraoperative images;
to coregister the portion of the patient's body to the preoperative imaging data within the navigation system common coordinate system, the coregistering including updating the preoperative imaging data by moving at least one of the segmented substructures relative to others of the segmented substructures; and
to display the current location of the surgical instrument with respect to the preoperative imaging data upon the output device, based upon the coregistering.

In some embodiments, the computer processor is configured, within less than 100 ms of the acquisition of the intraoperative images:
to identify the portion of the patient's body within the intraoperative images;
to segment the portion of the patient's body within the intraoperative images;
to perform 3D reconstruction of the portion of the patient's body based on at least some of the intraoperative images;
to coregister the portion of the patient's body to the preoperative imaging data within the navigation system common coordinate system, the coregistering including updating the preoperative imaging data by moving at least one of the segmented substructures relative to others of the segmented substructures; and
to display the current location of the surgical instrument with respect to the preoperative imaging data upon the output device, based upon the coregistering.

In some embodiments, the apparatus further includes the imaging system, the imaging system includes a stereoscopic RGB camera.

In some embodiments, the apparatus further includes the imaging system, and the imaging system includes a stereoscopic infrared camera.

In some embodiments, the apparatus further includes the imaging system, and the imaging system includes a combination of one or more RGB cameras and one or more infrared cameras.

In some embodiments, the apparatus further includes a light source, the computer processor is configured to perform 3D reconstruction of the portion of the patient's body based on at least some of the intraoperative images by:
driving the light source to direct light toward the portion of the patient's body; and
detecting light that is reflected from the portion of the patient's body within the intraoperative images.

In some embodiments, the light source includes a random structure laser light source that is configured to create a pattern of laser light on the portion of the patient's body.

In some embodiments, the apparatus further includes the imaging system, the imaging system includes a stereoscopic RGB camera, and the computer processor is configured to perform 3D reconstruction of the portion of the patient's body by calculating a disparity between images acquired by respective sensors within the stereoscopic RGB camera.

In some embodiments, the apparatus further includes the imaging system, the imaging system includes a stereoscopic infrared camera, and the computer processor is configured to perform 3D reconstruction of the portion of the patient's body by calculating a disparity between images acquired by respective sensors within the stereoscopic infrared camera.

In some embodiments, the apparatus further includes the imaging system, the imaging system includes a combination of one or more RGB cameras and one or more infrared cameras, and the computer processor is configured to perform 3D reconstruction of the portion of the patient's body by registering images acquired by the one or more RGB cameras to images acquired by the one or more infrared cameras.

In some embodiments, the computer processor is configured to receive preoperative planning that is performed with respect to the preoperative imaging data of the portion of the patient's body and the computer processor is configured to drive the output device to display a current location of the surgical instrument with respect to the preoperative planning, based upon the coregistering.

In some embodiments, the computer processor is configured to receive preoperative planning of a trajectory of the surgical instrument with respect to the preoperative imaging data of the portion of the patient's body and the computer processor is configured to drive the output device to display a current location of the surgical instrument with respect to the preoperative planning of the trajectory of the surgical instrument, based upon the coregistering.

In some embodiments, the computer processor is configured to receive preoperative planning of target tissue with respect to the preoperative imaging data of the portion of the patient's body and the computer processor is configured to drive the output device to display a current location of the surgical instrument with respect to the preoperative planning of the target tissue, based upon the coregistering.

In some embodiments, the computer processor is configured to receive preoperative planning of implantation of an implant with respect to the preoperative imaging data of the portion of the patient's body and the computer processor is configured to drive the output device to display a current location of the surgical instrument with respect to the preoperative planning of the implantation of the implant, based upon the coregistering.

In some embodiments, the computer processor is configured to receive preoperative planning of implantation of an electrode with respect to preoperative imaging data of brain tissue and the computer processor is configured to drive the output device to display a current location of the surgical instrument with respect to the preoperative planning of the implantation of the electrode with respect to the preoperative imaging data of brain tissue, based upon the coregistering.

In some embodiments, the computer processor is configured to receive preoperative planning of implantation of a cage with respect to preoperative imaging data of spinal tissue and the computer processor is configured to drive the output device to display a current location of the surgical instrument with respect to the preoperative planning of the implantation of the cage with respect to the preoperative imaging data of the spinal tissue, based upon the coregistering.

In some embodiments, the apparatus further includes the imaging system, the imaging system includes a hyperspectral camera, and the computer processor is configured to coregister the portion of the patient's body to the preoperative imaging data within the navigation system common coordinate system using imaging data acquired using the hyperspectral camera.

In some embodiments, the computer processor is configured to coregister the portion of the patient's body to the preoperative imaging data within the navigation system common coordinate system using spectral imaging data that are indicative of a given tissue type.

In some embodiments, the computer processor is configured to coregister the portion of the patient's body to the preoperative imaging data within the navigation system common coordinate system at least partially by deforming the portion of the patient's body within the preoperative imaging data.

In some embodiments, the computer processor is configured to coregister the portion of the patient's body to the preoperative imaging data within the navigation system common coordinate system at least partially by deforming the portion of the patient's body within the preoperative imaging data, using a non-rigid coregistration algorithm.

In some embodiments, the computer processor is configured to coregister the portion of the patient's body to the preoperative imaging data within the navigation system common coordinate system, by performing surface-matching registration between a surface of the portion of the patient's body as it appears within the preoperative imaging data and a current shape of the surface of portion of the patient's body.

In some embodiments, the computer processor is further configured to coregister the portion of the patient's body to the preoperative imaging data within the navigation system common coordinate system by modelling changes between the shapes of internal portions of the portion of the patient's body as the internal portions of the portion of the patient's body appear in preoperative imaging data and current shapes of internal portions of the portion of the patient's body appear, based upon the surface-matching registration and tissue that is present within the internal portions of the portion of the patient's body.

In some embodiments, the computer processor is further configured to coregister the portion of the patient's body to the preoperative imaging data within the navigation system common coordinate system by determining that the portion of the patient's body has been cut, and modifying the preoperative imaging data to create an accurate representation of the cut organ.

In some embodiments, the computer processor is configured to coregister the portion of the patient's body to the preoperative imaging data within the navigation system common coordinate system, subsequent to the portion of the patient's body having undergone movement, deformation and/or resection since the preoperative imaging data were acquired.

In some embodiments, the computer processor is configured to coregister the portion of the patient's body to the preoperative imaging data within the navigation system common coordinate system, while the portion of the patient's body undergoes intraprocedural movement, deformation and/or resection.

There is further provided, in accordance with some embodiments of the present invention, a method for use during a surgical procedure that is performed on a portion of a body of a patient using a surgical instrument, an imaging system, an output device, fiducial markers placed upon the patient's body, and a surgical navigation system that is configured to coregister anatomy of the patient with the preoperative imaging data of the portion of the patient's body such that the patient's anatomy and the preoperative imaging data are registered with each other within a navigation system common coordinate system, by identifying the fiducial markers within images of the patient's body, the method including:
using at least one computer processor:
   receiving preoperative imaging data of the portion of the patient's body and the surgical instrument;
   segmenting the preoperative imaging data of the portion of the subject's body into substructures; and
   during the surgical procedure:
      coregistering the segmented substructure to the patient's body, such that the patient's body and the segmented substructures within the preoperative imaging data are registered within a navigation system common coordinate system;
      coregistering images acquired by the imaging system within the navigation system common coordinate system;
      receiving intraoperative images of the portion of the patient's body from the imaging system;
      identifying the portion of the patient's body within the intraoperative images;
      segmenting the portion of the patient's body within the intraoperative images;
      performing 3D reconstruction of the portion of the patient's body based on at least some of the intraoperative images;
      coregistering the portion of the patient's body to the preoperative imaging data within the navigation system common coordinate system, the coregistering including updating the preoperative imaging data by moving at least one of the segmented substructures relative to others of the segmented substructures; and
      driving the output device to display a current location of the surgical instrument with respect to the preoperative imaging data, based upon the coregistering.

There is further provided, in accordance with some embodiments of the present invention, apparatus for use during a surgical procedure that is performed on a portion of a body of a patient using a surgical instrument, an imaging system, and an output device, the apparatus including:
at least one computer processor configured:
   to receive preoperative imaging data of the portion of the patient's body; and
   during the surgical procedure:
      to receive intraoperative images of the portion of the patient's body and the surgical instrument from the imaging system;
      to identify the portion of the patient's body within the intraoperative images;
      to segment the portion of the patient's body within the intraoperative images;
      to perform 3D reconstruction of the portion of the patient's body based on at least some of the intraoperative images;
      to coregister the portion of the patient's body to a common coordinate system with the portion of the patient's body as it appears within the preoperative imaging data using a non-rigid coregistration algorithm; and
      to drive the output device to display a current location of the surgical instrument with respect to the preoperative imaging data, based upon the coregistering.

In some embodiments, the computer processor is configured to coregister the portion of the patient's body to a common coordinate system with the portion of the patient's body as it appears within the preoperative imaging data by deforming the portion of the patient's body within the preoperative imaging data, using the non-rigid coregistration algorithm.

In some embodiments, in response to detecting that the portion of the patient's body has undergone a change in shape since the preoperative imaging data were acquired, the computer processor is configured to update a shape of the preoperative imaging data to reflect the change in shape that the portion of the patient's body has undergone.

In some embodiments, the computer processor is configured to drive the output device to display the current location of the surgical instrument with respect to the preoperative imaging data, based upon the coregistering, without requiring use of instrument fiducial markers disposed on the surgical instrument.

In some embodiments, the apparatus further includes the imaging system, the apparatus is configured for use with a surgical lighting system that includes a handle and at least a portion of the imaging system is disposed on a cover that is configured to be placed over the handle.

In some embodiments, the apparatus further includes the imaging system, the imaging system includes one or more infrared cameras that are configured to acquire images of the veins within the portion of the patient's body.

In some embodiments, the apparatus further includes the imaging system, and the imaging system includes one or more depth cameras.

In some embodiments, the computer processor is configured, in real time with respect to the acquisition of the intraoperative images:
to identify the portion of the patient's body within the intraoperative images;
to segment the portion of the patient's body within the intraoperative images;
to perform 3D reconstruction of the portion of the patient's body based on at least some of the intraoperative images;
to coregister the portion of the patient's body to a common coordinate system with the portion of the patient's body as it appears within the preoperative imaging data; and
to display the current location of the surgical instrument with respect to the preoperative imaging data upon the output device, based upon the coregistering.

In some embodiments, the computer processor is configured, within less than 100 ms of the acquisition of the intraoperative images:
to identify the portion of the patient's body within the intraoperative images;
to segment the portion of the patient's body within the intraoperative images;
to perform 3D reconstruction of the portion of the patient's body based on at least some of the intraoperative images;
to coregister the portion of the patient's body to a common coordinate system with the portion of the patient's body as it appears within the preoperative imaging data; and
to display the current location of the surgical instrument with respect to the preoperative imaging data upon the output device, based upon the coregistering.

In some embodiments, the apparatus further includes the imaging system, the imaging system includes a stereoscopic RGB camera.

In some embodiments, the apparatus further includes the imaging system, and the imaging system includes a stereoscopic infrared camera.

In some embodiments, the apparatus further includes the imaging system, and the imaging system includes a combination of one or more RGB cameras and one or more infrared cameras.

In some embodiments, the computer processor is configured to coregister the portion of the patient's body to the common coordinate system with the portion of the patient's body as it appears within the preoperative imaging data, by performing surface-matching registration between a surface of the portion of the patient's body as it appears within the preoperative imaging data and a current shape of the surface of portion of the patient's body.

In some embodiments, the computer processor is further configured to coregister the portion of the patient's body to the common coordinate system with the portion of the patient's body as it appears within the preoperative imaging data by modelling changes between the shapes of internal portions of the portion of the patient's body as the internal portions of the portion of the patient's body appear in preoperative imaging data and current shapes of internal portions of the portion of the patient's body appear, based upon the surface-matching registration and tissue that is present within the internal portions of the portion of the patient's body.

In some embodiments, the computer processor is further configured to coregister the portion of the patient's body to the common coordinate system with the portion of the patient's body as it appears within the preoperative imaging data by determining that the portion of the patient's body has been cut, and modifying the preoperative imaging data to create an accurate representation of the cut organ.

In some embodiments, the apparatus further includes a light source, and the computer processor is configured to perform 3D reconstruction of the portion of the patient's body based on at least some of the intraoperative images by:
driving the light source to direct light toward the portion of the patient's body; and
detecting light that is reflected from the portion of the patient's body within the intraoperative images.

In some embodiments, the light source includes a random structure laser light source that is configured to create a pattern of laser light on the portion of the patient's body.

In some embodiments, the apparatus further includes the imaging system, the imaging system includes a stereoscopic RGB camera, and the computer processor is configured to perform 3D reconstruction of the portion of the patient's body by calculating a disparity between images acquired by respective sensors within the stereoscopic RGB camera.

In some embodiments, the apparatus further includes the imaging system, the imaging system includes a stereoscopic infrared camera, and the computer processor is configured to perform 3D reconstruction of the portion of the patient's body by calculating a disparity between images acquired by respective sensors within the stereoscopic infrared camera.

In some embodiments, the apparatus further includes the imaging system, the imaging system includes a combination of one or more RGB cameras and one or more infrared cameras, and the computer processor is configured to perform 3D reconstruction of the portion of the patient's body by registering images acquired by the one or more RGB cameras to images acquired by the one or more infrared cameras.

In some embodiments, the computer processor is configured to receive preoperative planning that is performed with respect to the preoperative imaging data of the portion of the patient's body and the computer processor is configured to drive the output device to display a current location of the surgical instrument with respect to the preoperative planning, based upon the coregistering.

In some embodiments, the computer processor is configured to receive preoperative planning of a trajectory of the surgical instrument with respect to the preoperative imaging data of the portion of the patient's body and the computer processor is configured to drive the output device to display a current location of the surgical instrument with respect to the preoperative planning of the trajectory of the surgical instrument, based upon the coregistering.

In some embodiments, the computer processor is configured to receive preoperative planning of target tissue with respect to the preoperative imaging data of the portion of the patient's body and the computer processor is configured to drive the output device to display a current location of the surgical instrument with respect to the preoperative planning of the target tissue, based upon the coregistering.

In some embodiments, the computer processor is configured to receive preoperative planning of implantation of an implant with respect to the preoperative imaging data of the portion of the patient's body and the computer processor is configured to drive the output device to display a current location of the surgical instrument with respect to the preoperative planning of the implantation of the implant, based upon the coregistering.

In some embodiments, the computer processor is configured to receive preoperative planning of implantation of an electrode with respect to preoperative imaging data of brain tissue and the computer processor is configured to drive the output device to display a current location of the surgical instrument with respect to the preoperative planning of the implantation of the electrode with respect to the preoperative imaging data of brain tissue, based upon the coregistering.

In some embodiments, the computer processor is configured to receive preoperative planning of implantation of a cage with respect to preoperative imaging data of spinal tissue and the computer processor is configured to drive the output device to display a current location of the surgical instrument with respect to the preoperative planning of the implantation of the cage with respect to the preoperative imaging data of the spinal tissue, based upon the coregistering.

In some embodiments, the apparatus further includes the imaging system, the imaging system includes a hyperspectral camera, and the computer processor is configured to coregister the portion of the patient's body to a common coordinate system with the portion of the patient's body as it appears within the preoperative imaging data using imaging data acquired using the hyperspectral camera.

In some embodiments, the computer processor is configured to coregister the portion of the patient's body to a common coordinate system with the portion of the patient's body as it appears within the preoperative imaging data using spectral imaging data that are indicative of a given tissue type.

In some embodiments, wherein the computer processor is configured for use with fiducial markers placed upon the patient's body and a surgical navigation system that is configured to coregister anatomy of the patient with the preoperative imaging data of the portion of the patient's body such that the patient's anatomy and the preoperative imaging data are registered with each other within a navigation system common coordinate system, by identifying the fiducial markers within images of the patient's body.

In some embodiments, wherein the computer processor is configured to coregister images acquired by the imaging system within the navigation system common coordinate system.

In some embodiments, wherein the fiducial markers include fiducial markers that are visible within images acquired by the imaging system, and wherein the computer processor is configured to coregister images acquired by the imaging system within the navigation system common coordinate system by identifying the fiducial markers within images acquired by the imaging system.

In some embodiments, the apparatus further includes markers coupled to the imaging system, wherein the computer processor is configured to coregister images acquired by the imaging system within the navigation system common coordinate system by tracking the markers that are coupled to the imaging system.

In some embodiments, wherein in response to detecting that the portion of the patient's body has undergone a change in shape since the preoperative imaging data were acquired, the computer processor is configured to update a shape of the preoperative imaging data and registration of the preoperative imaging data within the navigation system common coordinate system to reflect the change in shape that the portion of the patient's body has undergone.

In some embodiments, wherein the computer processor is configured to coregister the portion of the patient's body to a common coordinate system with the portion of the patient's body as it appears within the preoperative imaging data, subsequent to the portion of the patient's body having undergone movement, deformation and/or resection since the preoperative imaging data were acquired.

In some embodiments, wherein the computer processor is configured to coregister the portion of the patient's body to a common coordinate system with the portion of the patient's body as it appears within the preoperative imaging data, while the portion of the patient's body undergoes intraprocedural movement, deformation and/or resection.

There is further provided, in accordance with some embodiments of the present invention, a method for use during a surgical procedure that is performed on a portion of a body of a patient using a surgical instrument, the apparatus including:
acquiring preoperative imaging data of the portion of the patient's body; and
during the surgical procedure:
   acquiring intraoperative images of the portion of the patient's body and the surgical instrument; and
   using at least one computer processor:
      identifying the portion of the patient's body within the intraoperative images;
      segmenting the portion of the patient's body within the intraoperative images;
      performing 3D reconstruction of the portion of the patient's body based on at least some of the intraoperative images;
      coregistering the portion of the patient's body to a common coordinate system with the portion of the patient's body as it appears within the preoperative imaging data using a non-rigid coregistration algorithm; and
      displaying a current location of the surgical instrument with respect to the preoperative imaging data upon an output device, based upon the coregistering.

The present invention will be more fully understood from the following detailed description of applications thereof, taken together with the drawings, in which:

### BRIEF DESCRIPTION OF THE DRAWINGS

FIG. 1A is a schematic illustration of a physician performing brain surgery on a patient using surgical navigation, in accordance with some applications of the present invention;

FIG. 1B is a schematic illustration of a physician performing brain surgery on a patient using surgical navigation, in accordance with some additional applications of the present invention;

FIG. 1C is a schematic illustration of a physician performing brain surgery on a patient using surgical navigation, in accordance with some further applications of the present invention;

FIG. 2 is a flowchart showing steps of a method at least some of which are typically performed in a surgical navigation procedure, in accordance with some applications of the present invention; and

FIG. 3 is a flowchart showing steps of a method at least some of which are typically performed in a surgical navigation procedure, in accordance with some alternative applications of the present invention.

### DETAILED DESCRIPTION OF EMBODIMENTS

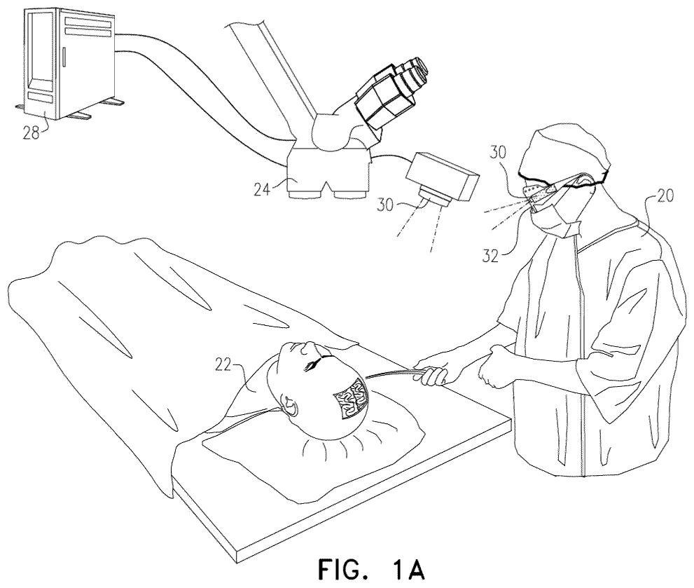

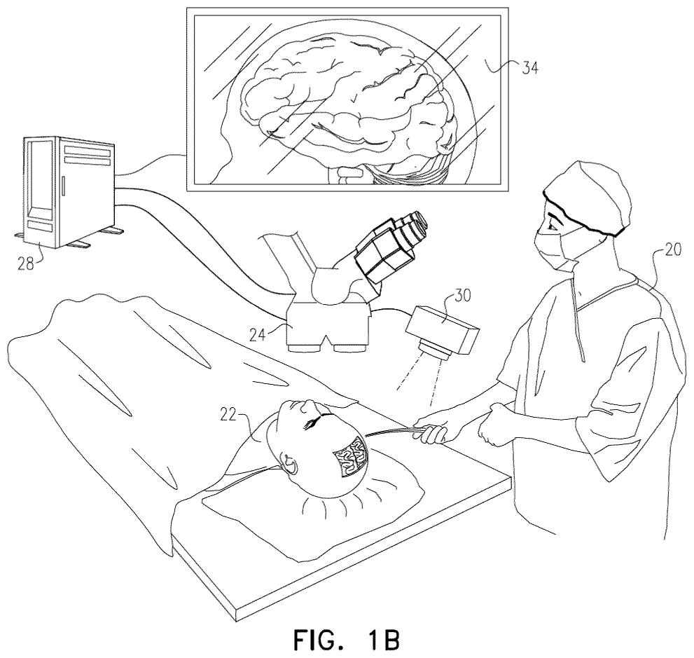

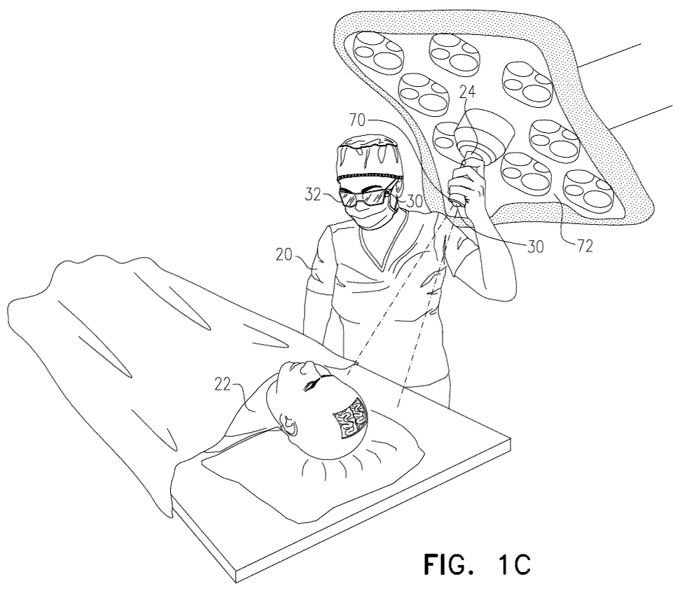

Reference is now made to FIG. 1A, which is a schematic illustration of a physician 20 performing brain surgery on a patient 22 using surgical navigation, in accordance with some applications of the present invention. Reference is also made to FIGS. 1B and 1C, which are schematic illustrations of physician 20 performing brain surgery on patient 22 using surgical navigation, in accordance with some additional applications of the present invention. Although FIGS. 1A-1C show the apparatus and methods being described herein being used during brain surgery, the scope of the present disclosure includes using the apparatus and methods described herein in the context of other surgical procedures, mutatis mutandis. The apparatus and methods described herein are particularly applicable to surgical procedures that are performed on non-rigid tissue and/or on tissue that is prone to undergo movement, deformation, and/or resection (for example, in the case of bone that is cut) either during the procedure and/or between a presurgical image-acquisition stage and the surgery itself.

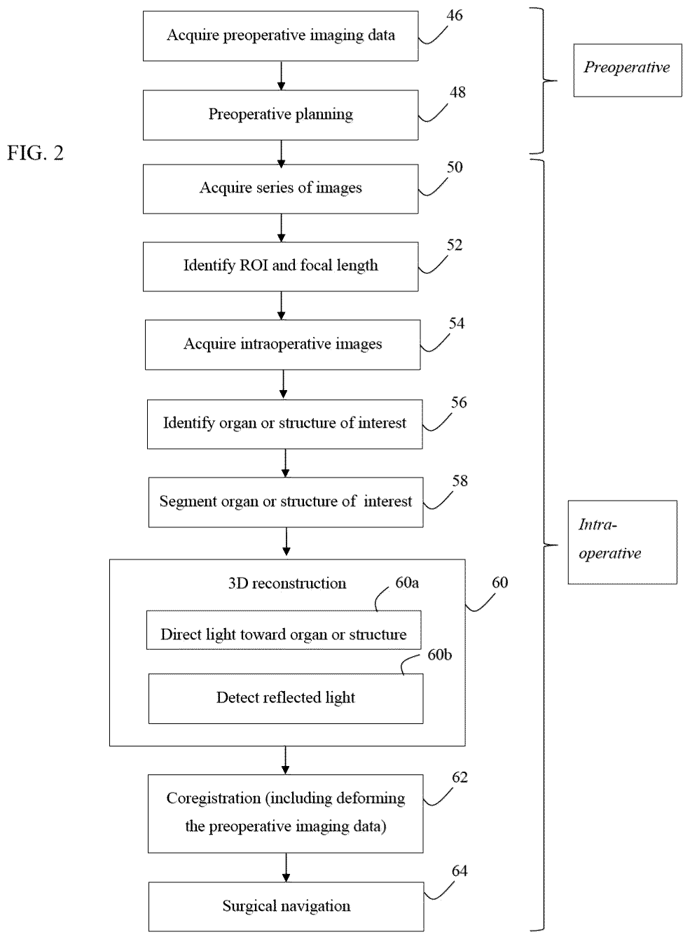

Reference is also now made to FIG. 2, which is a flowchart showing steps of a method at least some of which are typically performed in a surgical navigation procedure, in accordance with some applications of the present invention. It is noted that the particular series of steps shown in FIG. 2 is optional, and in some applications, some of the steps (such as steps 50 and 52) may be omitted. In general, the scope of the present disclosure includes performing only a portion of the steps shown in FIG. 2, whether in the order in which they are shown or in a different order, mutatis mutandis.

Typically, prior to the procedure being performed, preoperative imaging data are acquired (step 46). Typically, a 3D imaging modality is used to acquire the preoperative imaging data. For example, 3D CT, MRI, PET, PET-CT, radiographical, ultrasound images, and/or other types of images may be acquired. Alternatively or additionally, a 2D imaging modality is used to acquire the preoperative imaging data. For example, x-ray, ultrasound, MRI, and/or other types of images may be acquired. In some cases, additional preoperative data is utilized, for example non-patient-specific data, e.g., an anatomical atlas or other data that reflect known anatomical structures or parameters. For some applications, preoperative planning is performed with respect to the preoperative imaging data (step 48). For example, the trajectory of a surgical instrument through the patient's anatomy may be pre-planned using the preoperative imaging data. Alternatively or additionally, a target tissue, such as a lesion or a tumor may be located within the preoperative imaging data. For some applications, the preoperative planning includes planning the delivery and/or the deployment of an implant, for example, the implantation of an electrode in the brain, and/or the implantation of a cage (or other implant) in the spine.

FIGS. 1A-1C are schematic illustrations of the patient during surgery, with the step 46 (and optionally step 48) having already been performed. As described in the Background section, typically prior art surgical navigation techniques involve coregistering the patient's anatomy to the preoperative imaging data, such that corresponding points in the patient's anatomy and the preoperative imaging data are aligned with each other, within a common coordinate system. Typically, fiducial markers are placed on the patient's body as well as on the surgical instrument, such that the location of the surgical instrument with respect to the patient's anatomy is derived. By virtue of the coregistration of the patient's anatomy to the preoperative imaging data, the location of the surgical instrument with respect to the preoperative imaging data and/or with respect to the preoperative planning is thereby derived. Typically, in such cases, the fixation of fiducial markers on the patient must be rigid and the fiducial markers are placed in fixed position relative to the anatomy that is to be navigated. Furthermore, if the anatomy moves during surgery, or the fiducial markers move with respect to the patient, the coregistration procedure must be performed again. Moreover, if the anatomy that is to be treated changes during surgery (e.g., duc to tissue being cut, bones being broken, etc.), navigation cannot be used since preoperative imaging is no longer an accurate representation of the current anatomy.

Due to the aforementioned limitations, soft-tissue organs and tissue that is modified during surgery (e.g., due to tissue being cut, bones being broken, etc.) cannot be navigated with high accuracy using the above-described techniques. This is because such techniques rely upon the fiducial markers being maintained in rigidly-fixed positions with respect to the anatomy that is to be navigated, whereas soft tissue undergoes movement between the acquisition of the preoperative imaging date and the surgical procedure, and additionally undergoes movement during the surgical procedure (both as a result of natural movement as well as movement that is brought about by the interaction between the surgical instruments and the soft tissue). Therefore, in practice, surgical navigation is typically only performed on rigid anatomy such as bones, spine, and cars nose and throat (ENT). (In addition, surgical navigation is performed with respect to the lungs by using the network of airways as navigational guides.) In some cases, surgical navigation is performed in conjunction with brain surgery, based on the brain being encapsulated within the skull and therefore being held in a relatively fixed position. However, the brain sometimes move inside the skull when the skull is opened (in a phenomenon that is known as “brain shift”), and/or during surgery as a result of the surgery. Therefore, surgical navigation in brain surgery suffers from certain drawbacks. As described hereinabove, even in surgery that is performed with respect to rigid tissue, such as bones, the tissue often undergoes movement between the acquisition of the preoperative imaging date and the surgical procedure, and/or undergoes movement during the surgical procedure, e.g., as a result of bones being broken and/or moved, such that the coregistration becomes inaccurate.

Some applications of the present disclosure are directed toward overcoming the above-described limitations, such that surgical navigation is performed accurately with respect to soft tissue (e.g., organs such as the liver, spleen, or kidneys) and/or with respect to tissue that undergoes movement between the acquisition of the preoperative imaging date and the surgical procedure and/or undergoes movement during the surgical procedure (such as the brain). For example, some applications of the present disclosure are applied to operating upon vessels within the brain.

Referring again to FIGS. 1A-1C and 2, for some applications, the operating room includes an imaging system 24, such as a digital camera (and typically, a stereoscopic high-resolution camera). For some applications, the imaging system includes one or more red-green-blue (“RGB”) cameras, e.g., a pair of lenses and corresponding sensors arranged to provided stereoscopic vision. Alternatively or additionally, the imaging system includes one or more near-infrared (“NIR”) cameras, e.g., a pair of lenses and corresponding sensors arranged to provided stereoscopic vision. For some applications, a combination of RGB and NIR cameras is used, with the two types of camera typically working in parallel such as to increase the accuracy and/or efficiency of one or more of the image processing steps described hereinbelow. Typically, the cameras are pre-calibrated with respect to each other (e.g., when the cameras are assembled in the manufacturing process), such that each pixel in a given camera is coregistered with a corresponding pixel on the other cameras. For example, light that is detected by the NIR camera (e.g., light generated by a random structure laser light source as described hereinbelow) are automatically registered with pixels within the RGB cameras. Typically, this increases the accuracy and/or efficiency of one or more of the image processing steps described hereinbelow. For some applications, the imaging system includes a hyperspectral camera. For some applications, the imaging system includes a depth camera, such as a light detection and ranging (“LiDAR”) system.

For some applications, in an initial intraoperative step (step 50), the imaging system acquires a series of images at respective focal lengths. A computer processor 28 identifies the region of interest within the series of images and thereby sets the focal length to be used by the imaging system for the further imaging steps that are to be performed by the imaging system (step 52). As described hereinabove, for some applications the imaging system includes an NIR camera. For some applications, the NIR camera is used to acquire images of veins within a portion of the patient's body (since deoxygenated blood with hemoglobin typically forms a dark contrast on NIR images). For some applications, the method shown in FIG. 2 is performed without performing steps 50 and 52, i.e., with the intraoperative steps proceeding from step 54.

Typically, subsequent to the region of interest having been identified and the focal length derived, the imaging system acquires images of the surgical region of interest (step 54, FIG. 2). For some applications, computer processor 28 identifies a portion of interest within the images of the surgical region of interest (e.g., an organ of interest, such as the kidney or liver, or one or more structures within an organ that are of interest, e.g., a given vessel or set of vessels within the brain) using an object-detection algorithm (step 56, FIG. 2). For some applications, the computer processor runs an algorithm that has been pre-trained to identify the portion of the body. For example, the computer processor may run an algorithm that has pre-trained using machine-learning techniques, for example, a guided machine-learning algorithm, such as a convolutional neural network algorithm, using multiple real images of surgery with annotation of selected organs and structures. For some applications, the computer processor identifies the objects using a YOLO algorithm, an SSD algorithm, and/or an R-CNN algorithm. In a subsequent step (step 58), the computer processor performs segmentation of the identified organ and/or structures. Typically, the segmentation is applied such as to segment the identified organ and/or structures into substructures that that behave as semi-rigid substructures (such as the gyrus, vasculature within the brain, and/or abnormal structures, such as tumors, within the brain). For some applications, the computer processor performs instance segmentation of the identified organ and structures using a YOLO algorithm, e.g., an INSTA-YOLO algorithm. Alternatively or additionally, a different type of segmentation (e.g., an SSD algorithm, and/or an R-CNN algorithm) algorithm is used.

Subsequent to the identified organ and structures being segmented, 3D reconstruction (mapping) of only the segmented organs or structures is performed (step 60, FIG. 2). For some applications, the 3D reconstruction is performed by directing light toward the organ and/or structures of interest (step 60a) and detecting light reflected from the organ and/or structures using stereo vision and calculating the disparity between two sensors, e.g., the two sensors of a stereoscopic RGB camera, and/or the two sensors of a stereoscopic infrared camera (step 60b). For some applications, step 60a is performed by a laser light source 30 (e.g., a random structure laser light source, shown in FIGS. 1A-1C) creating a pattern of laser light on the organ and/or structures. Typically, the laser light is visible light (which is configured to be captured by RGB cameras) and/or NIR laser light (configured to be captured by NIR cameras). As described hereinabove, typically imaging system 24 includes a stereoscopic high-resolution camera. For some applications, the stereoscopic high-resolution camera includes RGB and/or NIR cameras. For some applications, the stereoscopic high-resolution camera is used to perform step 60b. For some applications, the distance between the two lenses of the camera is relatively small (e.g., less than 60 mm), for example, in order to apply the techniques described herein to procedures that include small incisions being made and/or minimally invasive surgeries (which are typically performed via a narrow cannula). Typically, in order to perform the 3D reconstruction, the distance between the camera and the organ and/or structures of interest in less than 100 cm. For some such applications, the camera is a microscopic camera. For some applications, the imaging system includes a depth camera, such as a light detection and ranging (“LiDAR”) system, and the 3D reconstruction is performed using the depth camera.

As described hereinabove, for some applications, the imaging system includes one or more red-green-blue (“RGB”) cameras, e.g., a pair of cameras arranged to provided stereoscopic vision. For some applications, the color-related data in the images acquired by the cameras are used in one or more of the object identification (step 56), object segmentation (step 58), and/or 3D reconstruction (step 60) algorithms. The use of color-related data in such algorithms typically adds more data as input to the algorithms (as opposed to using monochrome data, e.g. a monochrome depth camera such as in LiDAR), thereby typically increasing the speed and accuracy of these algorithms. Also as described hereinabove, for some applications, the imaging system includes one or more NIR cameras, e.g., a pair of cameras arranged to provided stereoscopic vision. For some applications, a combination of RGB and NIR cameras is used, with the two types of camera typically working in parallel such as to increase the accuracy and/or efficiency of one or more of the image-processing steps described hereinbelow. Typically, the cameras are pre-calibrated with respect to each other (e.g., when the cameras are assembled in the manufacturing process), such that each pixel in a given camera is coregistered with a corresponding pixel on the other cameras. For example, light that is detected by the NIR camera (e.g., light generated by the random structure laser light source) are automatically registered with pixels within the RGB cameras. Typically, this increases the accuracy and/or efficiency of one or more of the image processing steps described herein. For some applications, the NIR camera is used to acquire images of veins within a portion of the patient's body (since deoxygenated blood with hemoglobin typically forms a dark contrast on NIR images).

Subsequent to the 3D reconstruction of the organ and/or structures of interest, the organ and/or structures of interest are coregistered to the preoperative imaging data (step 62). The coregistration is typically performed using a coregistration algorithm that is applicable to non-rigid bodies. Typically, the coregistration is performed using surface-matching registration method of non-rigid bodies. For some applications, the coregistration includes a step of deforming the preoperative imaging data to match current position and shape of the organ and/or structure of interest. For example, the preoperative imaging data (e.g., CT and/or MRI imaging data) of an organ may include data relating to the shape of a soft tissue organ which is different than the intraoperative shape of the organ in surgery. In addition, during the procedure the shape of the organ may undergo changes (e.g., due to natural movement, due to movement of the organ by the surgical instruments, and/or due to the organ being cut). Typically, in such cases, the coregistration includes a step of deforming the preoperative imaging data to match current position and shape of the organ and/or structure of interest. Typically, the computer processor determines how to deform the preoperative imaging data by (a) performing surface-matching registration to determine how to deform the surface of the organ within the preoperative imaging data, and (b) modeling how the change in shape of the surface of the organ affects the shape of internal portions of the organ, based on mechanical models of behavior of the tissue that is present within the organ. For example, internal portions of the liver and brain will deform less than those of the bowels, while nerves will in some cases deform the most. Typically, by knowing the relationship between the surface of a given organ and the internal portions of the organ, the computer processor is able to accurately model how to deform the whole organ based upon the surface-matching registration.

It is noted that, as described in the above paragraph, typically in cases in which an organ or a portion thereof is cut, then as part of the coregistration step, the preoperative imaging data is modified to create an accurate representation of the cut organ (typically, by removing parts of the preoperative imaging data corresponding to the part that has been cut). Typically, this increases the accuracy of the coregistration.

As described hereinabove, for some applications, the computer processor determines how to deform the preoperative imaging data by modeling how the change in shape of the surface of the organ affects the shape of internal portions of the organ, based on mechanical models of behavior of the tissue that is present within the organ. For some applications, by performing many such procedures, a machine-learning algorithm is trained such as to learn how the change in shape of the surface of the organ affects the shape of internal portions of the organ. In further procedures, the computer processor applies the trained algorithm such as to model how the change in shape of the surface of the organ will have affected the shape of internal portions of the organ within the procedure.

For some applications, the imaging system includes a hyperspectral camera, and imaging data acquired using the hyperspectral camera are used to perform the coregistration of intraoperative imaging data with preoperative imaging data. For example, spectral imaging data that are indicative of a given tissue type may be used to perform the coregistration. For some applications, data acquired using the hyperspectral camera are used to perform one or more additional steps of the procedure.

Typically once the coregistration has been performed, the current location of a surgical instrument with respect to the preoperative imaging data is displayed. Further typically, the physician navigates surgical instruments through the patient's anatomy, using the updated preoperative imaging data and/or preoperative planning to navigate (step 64). For some applications, the above-described steps are performed without requiring any fiducial markers to be disposed on the surgical instrument, such that the current location of a surgical instrument with respect to the preoperative imaging data is displayed even without any fiducial markers being disposed on the surgical instrument. Alternatively, for some applications, tool fiducial markers are disposed on the surgical instrument.

Referring again to FIG. 1A, for some applications, the current location of the surgical instrument with respect to the preoperative imaging data and/or preoperative planning is displayed on a head-up display, e.g., on the physician's eyewear 32 (e.g., augmented-reality glasses). Alternatively or additionally, the current location of the surgical instrument with respect to the preoperative imaging data and/or preoperative planning is displayed on a screen 34, as shown in FIG. 1B. For some applications, imaging system 24 and/or laser light source 30 are disposed on the physician's eyewear. Alternatively or additionally, imaging system 24 and/or laser light source 30 are disposed above the patient's body, e.g. on an overhead stand or on a gantry. For some applications, imaging system 24 and/or laser light source 30 are mounted on a dedicated mounting device, such as an articulated arm, a stand, a pole, a mounting device that is coupled to the surgical table, and/or a Mayfield® cranial stabilization device. (FIG. 1A schematically illustrates an example in which laser light sources are disposed both on the physician's eyewear and overhead.) Further alternatively or additionally, imaging system 24 and/or laser light source 30 are disposed on a surgical microscope. For some applications, the apparatus and methods are described herein are performed in conjunction with endoscopic and/or laparoscopic procedures. For some such applications, imaging system 24 and/or laser light source 30 are disposed on the endoscope or the laparoscope, respectively. Referring to FIG. 1C, for some applications, imaging system 24 and/or laser light source 30 are disposed on a cover 70 that is configured to be placed over the handle of a surgical lighting system 72. In accordance with respective applications, the handle cover is reusable or is disposable. Typically, the handle cover is sterile.

With reference to steps 56-60 of FIG. 2, it is noted that, by first identifying and segmenting the organ and/or structures of interest, and then only performing the 3D reconstruction with respect to the organ and/or structures of interest, the time and computational resources that are required for the 3D reconstruction are reduced relative to if the 3D reconstruction was to be performed with respect to a larger portion of the patient's body (e.g., the entire field of view of the image). In addition, it is typically the case that the same stereoscopic high-resolution RGB camera is used to acquire the images in which the objects are identified and then segmented, and then to acquire the images that are used for the 3D reconstruction, which facilitates rapid object identification, segmentation, and 3D reconstruction. Typically, the above-described features of the system described herein allows steps 54-62 (i.e., image acquisition, object identification, object segmentation, 3D reconstruction, and coregistration) to be performed in real-time (e.g., in less than 100 ms, less than 30 ms, or less than 20 ms, e.g., approximately 16 ms). In turn, the performance of these steps within such a small time frame allows the coregistration to be performed at relatively high frequency during the procedure, such that, even as the organ and/or structures of interest undergo movement during the procedure (and even if the organ and/or structures of interest have undergone movement since the acquisition of the preoperative imaging data, e.g., due to brain shift), the coregistration accurately reflects the current shape and position of the organ and/or structures of interest.

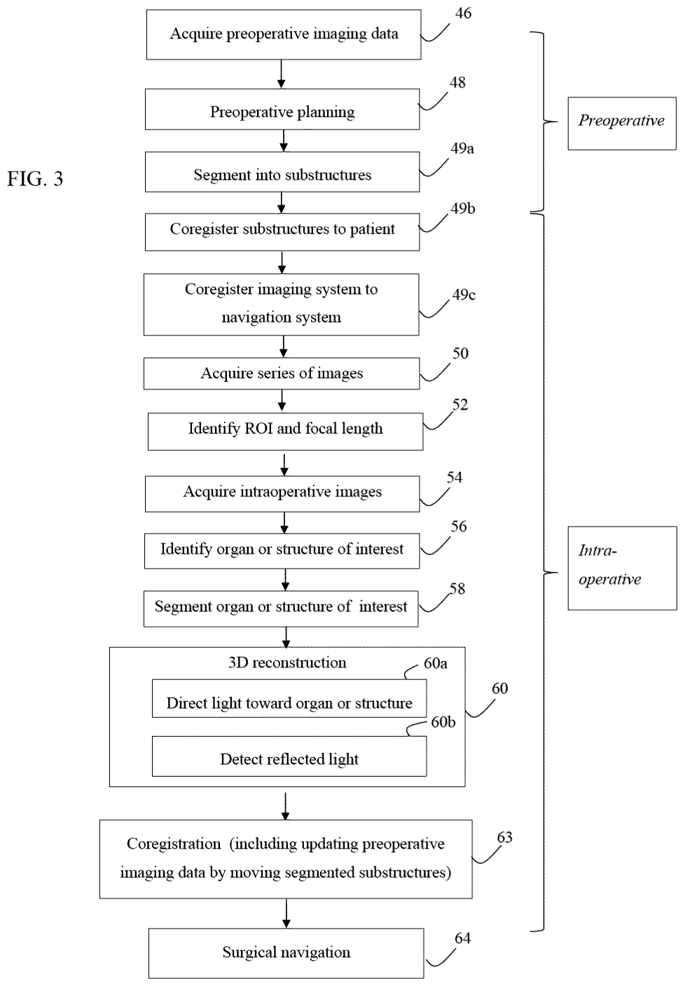

Reference is also now made to FIG. 3, which is a flowchart showing steps of a method at least some of which are typically performed in a surgical navigation procedure, in accordance with some applications of the present invention. Those steps that are shown in FIG. 3 with the same reference numerals as in FIG. 2 are performed in a generally similar manner to that described with reference to FIG. 2. In general, the method shown and described with reference to FIG. 3 differs from that shown and described with reference to FIG. 2 in terms of how the coregistration of the preoperative imaging data to the patient is performed, and in particular in terms of how the preoperative imaging data are updated to reflect changes in the tissue (e.g., movement, deformation and/or resection) that occur during the procedure. Typically, steps 49a, 49b, 49c and 63 are performed in order to carry out the coregistration. As noted with reference to FIG. 2, the particular series of steps shown in FIG. 3 is optional, and in some applications, some of the steps (such as steps 50 and 52) may be omitted. In general, the scope of the present disclosure includes performing only a portion of the steps shown in FIG. 3, whether in the order in which they are shown or in a different order, mutatis mutandis. For some applications, steps that are described with reference to FIGS. 2 and 3 are combined, as described in further details hereinbelow.

Typically, prior to the procedure being performed, preoperative imaging data are acquired (step 46). For some applications, preoperative planning is performed with respect to the preoperative imaging data (step 48). Steps 46 and 48 are typically performed in a generally similar manner to that described with reference to FIG. 2.

As described hereinabove, prior art surgical navigation techniques involve coregistering the patient's anatomy to the preoperative imaging data (e.g., using the Digital Imaging and Communications in Medicine (“DICOM®”) Standard), such that corresponding points in the patient's anatomy and the preoperative imaging data are registered with each other, within a navigation system common coordinate system. Typically, fiducial markers are placed on the patient's body as well as on the surgical instrument, such that the location of the surgical instrument with respect to the patient's anatomy is derived. By virtue of the coregistration of the patient's anatomy to the preoperative imaging data, the location of the surgical instrument with respect to the preoperative imaging data and/or with respect to the preoperative planning is thereby derived. For some applications, the prior art surgical navigation techniques are used to provide an initial estimate of the position of the organ, and/or a region of interest within the organ, relative to the preoperative imaging.

For some applications, within the preoperative imaging data, an organ is segmented into substructures, with respective datasets being created for each of the substructures within the navigation system common coordinate system (step 49a). Typically, the segmentation is applied such as to segment the identified organ into substructures that behave as semi-rigid sub-structures (such as the gyrus, vasculature within the brain, and/or abnormal structures, such as tumors, within the brain). For some applications, the computer processor performs instance segmentation of the identified organ and structures using a YOLO algorithm, e.g., an INSTA-YOLO algorithm. Alternatively or additionally, a different type of segmentation algorithm (e.g., an SSD algorithm, and/or an R-CNN algorithm) is used. Subsequently, the coregistration of the preoperative imaging data to the navigation system common coordinate system is performed (step 49b). Typically, the datasets for each of the substructures is coregistered to the same set of reference points within the navigation system common coordinate system (i.e., the fiducial markers on the patient), but the coregistration is performed separately for each of the substructures.

Typically, imaging system 24 (which typically includes an RGB camera and/or an IR camera) is coregistered with the navigation system (step 49c). Typically, each pixel within an image acquired by the imaging system is registered within the common coordinate system of the navigation system, such that images acquired by the imaging system are registered within the common coordinate system of the navigation system. For some applications, in order to facilitate the coregistration of the imaging system with the navigation system, fiducials that are placed on the patient's body (for use by the navigation system) are visible within images acquired by the imaging system. For example, the fiducials may be reflective (e.g., optically-reflective and/or IR-reflective) markers, for example, reflective (e.g., optically-reflective and/or IR-reflective) spheres. Alternatively or additionally, in order to facilitate the coregistration of the imaging system with the navigation system, the imaging system is tracked by a tracker (e.g., an electromagnetic tracker) of the navigation system using markers coupled to the imaging system.

Referring again to FIGS. 1A-1C, for some applications, the operating room includes an imaging system 24, such as a digital camera (and typically, a stereoscopic high-resolution camera). Typically, the imaging system is as described hereinabove. For some applications, the imaging system includes one or more red-green-blue (“RGB”) cameras, e.g., a pair of lenses and corresponding sensors arranged to provided stereoscopic vision. Alternatively or additionally, the imaging system includes one or more near-infrared (“NIR”) cameras, e.g., a pair of lenses and corresponding sensors arranged to provided stereoscopic vision. For some applications, a combination of RGB and NIR cameras is used, with the two types of camera typically working in parallel such as to increase the accuracy and/or efficiency of one or more of the image processing steps described hereinbelow. Typically, the cameras are pre-calibrated with respect to each other (e.g., when the cameras are assembled in the manufacturing process), such that each pixel in a given camera is coregistered with a corresponding pixel on the other cameras. For example, light that is detected by the NIR camera (e.g., light generated by a random structure laser light source as described hereinbelow) are automatically registered with pixels within the RGB cameras. Typically, this increases the accuracy and/or efficiency of one or more of the image processing steps described hereinbelow. For some applications, the imaging system includes a hyperspectral camera.

For some applications, in an initial intraoperative step (step 50), the imaging system acquires a series of images at respective focal lengths. A computer processor 28 identifies the region of interest within the series of images and thereby sets the focal length to be used by the imaging system for the further imaging steps that are to be performed by the imaging system (step 52). Steps 50 and 52 are typically performed in a generally similar manner to that described hereinabove with reference to FIG. 2. As described hereinabove, for some applications the imaging system includes an NIR camera. For some applications, the NIR camera is used to acquire images of veins within a portion of the patient's body (since deoxygenated blood with hemoglobin typically forms a dark contrast on NIR images). As noted with reference to FIG. 2, for some applications, the method shown in FIG. 3 is performed without performing steps 50 and 52, i.e., with the intraoperative steps proceeding from step 54.

Typically, subsequent to the region of interest having been identified and the focal length derived, the imaging system acquires images of the surgical region of interest (step 54). For some applications, computer processor 28 identifies a portion of interest within the images of the surgical region of interest (e.g., an organ of interest, such as the kidney or liver, or one or more structures within an organ that are of interest, e.g., a given vessel or set of vessels within the brain) using an object-detection algorithm (step 56). For some applications, the computer processor runs an algorithm that has been pre-trained to identify the portion of the body. For example, the computer processor may run an algorithm that has pre-trained using machine-learning techniques, for example, a guided machine-learning algorithm, such as a convolutional neural network algorithm, using multiple real images of surgery with annotation of selected organs and structures. For some applications, the computer processor identifies the objects using a YOLO algorithm, an SSD algorithm, and/or an R-CNN algorithm. In a subsequent step (step 58), the computer processor performs segmentation of the identified organ and/or structures. Typically, the segmentation is applied such as to segment the identified organ and/or structures into substructures that that behave as semi-rigid substructures (such as the gyrus, vasculature within the brain, and/or abnormal structures, such as tumors, within the brain). For some applications, the computer processor performs instance segmentation of the identified organ and structures using a YOLO algorithm, e.g., an INSTA-YOLO algorithm. Alternatively or additionally, a different type of segmentation algorithm (e.g., an SSD algorithm, and/or an R-CNN algorithm) is used. Steps 54, 56, and 58 are typically performed in a generally similar manner to that described hereinabove with reference to FIG. 2.

Subsequent to the identified organ and structures being segmented, 3D reconstruction (mapping) of only the segmented organs or structures is performed (step 60). Step 60 (including steps 60a and 60b) is typically performed in a generally similar manner to that described hereinabove with reference to FIG. 2. For some applications, the 3D reconstruction is performed by directing light toward the organ and/or structures of interest (step 60a) and detecting light reflected from the organ and/or structures using stereo vision and calculating the disparity between two sensors, e.g., the two sensors of a stereoscopic RGB camera, and/or the two sensors of a stereoscopic infrared camera (step 60b). For some applications, the imaging system includes a depth camera, such as a light detection and ranging (“LIDAR”) system, and the 3D reconstruction is performed using the depth camera.

Subsequent to the 3D reconstruction of the organ and/or structures of interest, the organ and/or structures of interest are coregistered to the preoperative imaging data within the navigation system common coordinate system (step 63). Coregistration step 63 differs from coregistration step 62 described with reference to FIG. 2. The coregistration is typically performed by coregistering the organ and/or structures of interest within the images to navigation system coordinate system, using the techniques described hereinabove with reference to step 49c. For some applications, during the procedure, in response to detecting that segmented substructures have moved relative to other segmented substructures, the preoperative imaging data are updated for use within the surgical navigation system. Typically, the shape of the preoperative imaging data is updated and the registration of the preoperative imaging data within the navigation system common coordinate system is updated to reflect movement of segmented substructures relative to other segmented substructures to reflect changes in the tissue (e.g., due to movement, deformation and/or resection), such that the reshaped organ is coregistered within the navigation system common coordinate system. Typically, the shape of the preoperative imaging data is updated and the registration of the preoperative imaging data within the navigation system common coordinate system is updated only with respect to substructures with respect to which a change of shape has been detected. In this manner, the updating of the shape and the coregistration is performed with respect to relatively small volumes of data, rather than an entire organ, thereby reducing computational resources, increasing the speed of the updating of the shape and the coregistration, and enhancing accuracy of the updating of the shape and the coregistration, relative to if these steps were performed with respect to the entire organ.

For some applications, the shape of the preoperative imaging data is updated and the registration of the preoperative imaging data within the navigation system common coordinate system is performed using a combination of the steps described with reference to FIGS. 2 and 3. For example, the preoperative imaging data is updated (a) to incorporate to reflect movement of segmented substructures relative to other segmented substructures to reflect changes in the tissue, in accordance with step 63 of FIG. 3, and in addition (b) to deform the preoperative imaging data within the segmented substructures, in accordance with step 62 of FIG. 2. As described with reference to step 62 of FIG. 2, for some applications, the computer processor determines how to deform the preoperative imaging data within the segmented substructures by (a) performing surface-matching registration to determine how to deform the surface of the organ within the preoperative imaging data, and (b) modeling how the change in shape of the surface of the organ affects the shape of internal portions of the organ, based on mechanical models of behavior of the tissue that is present within the organ.

Typically, in cases in which an organ or a portion thereof is cut, then as part of the coregistration step, the preoperative imaging data is modified to create an accurate representation of the cut organ (typically, by removing parts of the preoperative imaging data corresponding to the part that has been cut). Typically, this increases the accuracy of the coregistration.

For some applications, the imaging system includes a hyperspectral camera, and imaging data acquired using the hyperspectral camera are used to perform the coregistration of intraoperative imaging data with preoperative imaging data. For example, spectral imaging data that are indicative of a given tissue type may be used to perform the coregistration. For some applications, data acquired using the hyperspectral camera are used to perform one or more additional steps of the procedure.

Typically once the coregistration has been performed, the current location of a surgical instrument with respect to the preoperative imaging data is displayed. Further typically, the physician navigates surgical instruments through the patient's anatomy, using the updated preoperative imaging data and/or preoperative planning to navigate (step 64). For some applications, the above-described steps are performed without requiring any fiducial markers to be disposed on the surgical instrument, such that the current location of a surgical instrument with respect to the preoperative imaging data is displayed even without any fiducial markers being disposed on the surgical instrument. Alternatively, for some applications, tool fiducial markers are disposed on the surgical instrument.

For some applications, apparatus and methods described with reference to FIG. 2 are integrated with prior art surgical navigation techniques, for example, in the following manner. As described hereinabove, prior art surgical navigation techniques involve coregistering the patient's anatomy to the preoperative imaging data (e.g., using the DICOM® standard), such that corresponding points in the patient's anatomy and the preoperative imaging data are registered with each other, within a navigation system common coordinate system. Typically, fiducial markers are placed on the patient's body as well as on the surgical instrument, such that the location of the surgical instrument with respect to the patient's anatomy is derived. By virtue of the coregistration of the patient's anatomy to the preoperative imaging data, the location of the surgical instrument with respect to the preoperative imaging data and/or with respect to the preoperative planning is thereby derived.

For some applications, the prior art surgical navigation techniques are used to provide an initial estimate of the position of the organ, and/or a region of interest within the organ, relative to the preoperative imaging. Typically, imaging system 24 (which typically includes an RGB camera and/or an IR camera) is coregistered with the navigation system. Typically, each pixel within an image acquired by the imaging system is registered within the common coordinate system of the navigation system, such that images acquired by the imaging system are registered within the common coordinate system of the navigation system. For some applications, in order to facilitate the coregistration of the imaging system with the navigation system, fiducials that are placed on the patient's body (for use by the navigation system) are visible within images acquired by the imaging system. For example, the fiducials may be reflective (e.g., IR-reflective) markers, for example, reflective (e.g., IR-reflective) spheres. Alternatively or additionally, in order to facilitate the coregistration of the imaging system with the navigation system, the imaging system is tracked by a tracker (e.g., an electromagnetic tracker) of the navigation system using markers coupled to the imaging system.

For some applications, during the procedure, in response to detecting changes in the shape of the tissue (e.g., movement, deformation and/or resection), the preoperative imaging data is updated for use within the surgical navigation system. Typically, the shape of the preoperative imaging data is updated and the registration of the preoperative imaging data within the navigation system common coordinate system is updated to reflect changes in the shape of the tissue detected by the system, such that the reshaped organ is coregistered within the navigation system common coordinate system. For some applications, within the preoperative imaging data, an organ is segmented into substructures, with respective datasets being created for each of the substructures within the navigation system common coordinate system. Typically, each of the datasets is coregistered to the same set of reference points within the navigation system common coordinate system (i.e., the fiducial markers on the patient), but the coregistration is performed separately for each of the substructures. As described above, for some applications, during the procedure, the shape of the preoperative imaging data is updated and the registration of the preoperative imaging data to fiducials on the patient's body is updated to reflect changes in the shape of the tissue detected by the system. For some such applications, the shape of the preoperative imaging data is updated and the registration of the preoperative imaging data within the navigation system common coordinate system is updated only with respect to substructures with respect to which a change of shape has been detected. In this manner, the updating of the shape and the coregistration is performed with respect to relatively small volumes of data, rather than an entire organ, thereby reducing computational resources, increasing the speed of the updating of the shape and the coregistration, and enhancing accuracy of the updating of the shape and the coregistration, relative to if these steps were performed with respect to the entire organ.

With reference to computer processor 28, it is noted that although the computer processor is schematically illustrated as being a device within the operating room, the scope of the present disclosure includes any one of the steps described herein being performed by one or more remote computer processors that perform some of the algorithms described herein and that communicate with a local computer processor via a communications network. For some applications, a computer processor is built into the physician's eyewear and the computer processor performs one or more of the steps described herein. For some applications, a computer processor that is built into the physician's eyewear communicates with one or more remote computer processors via a communications network, and the remote computer processors perform one or more of the steps described herein. For some applications, a computer processor is built into imaging system 24 and the computer processor performs one or more of the steps described herein. For some applications, a computer processor that is built into the imaging system communicates with one or more remote computer processors via a communications network, and the remote computer processors perform one or more of the steps described herein.

Although some applications of the present disclosure have been described as being related to a procedure that is performed on a patient's brain, the scope of the present invention includes applying the apparatus and methods described herein to other portions of a patient's body, mutatis mutandis.

Applications of the invention described herein can take the form of a computer program product accessible from a computer-usable or computer-readable medium (e.g., a non-transitory computer-readable medium) providing program code for use by or in connection with a computer or any instruction execution system, such as computer processor 28. For the purpose of this description, a computer-usable or computer readable medium can be any apparatus that can comprise, store, communicate, propagate, or transport the program for use by or in connection with the instruction execution system, apparatus, or device. The medium can be an electronic, magnetic, optical, electromagnetic, infrared, or semiconductor system (or apparatus or device) or a propagation medium. Typically, the computer-usable or computer readable medium is a non-transitory computer-usable or computer readable medium.

Examples of a computer-readable medium include a semiconductor or solid-state memory, magnetic tape, a removable computer diskette, a random-access memory (RAM), a read-only memory (ROM), a rigid magnetic disk and an optical disk. Current examples of optical disks include compact disk-read only memory (CD-ROM), compact disk-read/write (CD-R/W) and DVD.

A data processing system suitable for storing and/or executing program code will include at least one processor (e.g., computer processor 28) coupled directly or indirectly to memory elements through a system bus. The memory elements can include local memory employed during actual execution of the program code, bulk storage, and cache memories which provide temporary storage of at least some program code in order to reduce the number of times code must be retrieved from bulk storage during execution. The system can read the inventive instructions on the program storage devices and follow these instructions to execute the methodology of the embodiments of the invention.

Network adapters may be coupled to the processor to enable the processor to become coupled to other processors or remote printers or storage devices through intervening private or public networks. Modems, cable modem and Ethernet cards are just a few of the currently available types of network adapters.

Computer program code for carrying out operations of the present invention may be written in any combination of one or more programming languages, including an object-oriented programming language such as Java, Smalltalk, C++ or the like and conventional procedural programming languages, such as the C programming language or similar programming languages.

It will be understood that blocks of the flowcharts shown in the figures and combinations of blocks in the flowcharts, can be implemented by computer program instructions. These computer program instructions may be provided to a processor of a general-purpose computer, special purpose computer, or other programmable data processing apparatus to produce a machine, such that the instructions, which execute via the processor of the computer (e.g., computer processor 28) or other programmable data processing apparatus, create means for implementing the functions/acts specified in the flowcharts and/or algorithms described in the present application. These computer program instructions may also be stored in a computer-readable medium (e.g., a non-transitory computer-readable medium) that can direct a computer or other programmable data processing apparatus to function in a particular manner, such that the instructions stored in the computer-readable medium produce an article of manufacture including instruction means which implement the function/act specified in the flowchart blocks and algorithms. The computer program instructions may also be loaded onto a computer or other programmable data processing apparatus to cause a series of operational steps to be performed on the computer or other programmable apparatus to produce a computer implemented process such that the instructions which execute on the computer or other programmable apparatus provide processes for implementing the functions/acts specified in the flowcharts and/or algorithms described in the present application.

Computer processor 28 is typically a hardware device programmed with computer program instructions to produce a special purpose computer. For example, when programmed to perform the algorithms described with reference to the figures, computer processor 28 typically acts as a special purpose surgical-navigation computer processor. Typically, the operations described herein that are performed by computer processor 28 transform the physical state of a memory, which is a real physical article, to have a different magnetic polarity, electrical charge, or the like depending on the technology of the memory that is used. For some applications, operations that are described as being performed by computer processor 28 are performed by a plurality of computer processors in combination with each other. For example, as described hereinabove, the scope of the present disclosure includes any one of the steps described herein being performed by one or more remote computer processors that perform some of the algorithms described herein and that communicate with a local computer processor via a communications network. For some applications, a computer processor is built into the physician's eyewear and the computer processor performs one or more of the steps described herein. For some applications, a computer processor that is built into the physician's eyewear communicates with one or more remote computer processors via a communications network, and the remote computer processors perform one or more of the steps described herein. For some applications, a computer processor is built into imaging system 24 and the computer processor performs one or more of the steps described herein. For some applications, a computer processor that is built into the imaging system communicates with one or more remote computer processors via a communications network, and the remote computer processors perform one or more of the steps described herein.

It will be appreciated by persons skilled in the art that the present invention is not limited to what has been particularly shown and described hereinabove. Rather, the scope of the present invention includes both combinations and subcombinations of the various features described hereinabove, as well as variations and modifications thereof that are not in the prior art, which would occur to persons skilled in the art upon reading the foregoing description.

1. An apparatus for use during a surgical procedure that is performed on a portion of a body of a patient using a surgical instrument, an imaging system, and an output device, the apparatus comprising:
at least one computer processor configured:
to receive preoperative imaging data of the portion of the patient's body; and
during the surgical procedure:
to receive intraoperative images of the portion of the patient's body and the surgical instrument from the imaging system;
to identify the portion of the patient's body within the intraoperative images;
to segment the portion of the patient's body within the intraoperative images;
to perform 3D reconstruction of the portion of the patient's body based on at least some of the intraoperative images;
to coregister the portion of the patient's body to a common coordinate system with the portion of the patient's body as it appears within the preoperative imaging data using a non-rigid coregistration algorithm; and
to drive the output device to display an output, based upon the coregistering.

2. The apparatus according to claim 1, wherein the computer processor is configured to coregister the portion of the patient's body to a common coordinate system with the portion of the patient's body as it appears within the preoperative imaging data by deforming the portion of the patient's body within the preoperative imaging data, using the non-rigid coregistration algorithm.

3. The apparatus according to claim 1, wherein in response to detecting that the portion of the patient's body has undergone a change in shape since the preoperative imaging data were acquired, the computer processor is configured to update a shape of the preoperative imaging data to reflect the change in shape that the portion of the patient's body has undergone.

4. The apparatus according to claim 1, wherein the computer processor is configured to drive the output device to display a current location of the surgical instrument with respect to the preoperative imaging data, based upon the coregistering.

5. The apparatus according to claim 4, wherein the computer processor is configured to drive the output device to display the current location of the surgical instrument with respect to the preoperative imaging data, based upon the coregistering, without requiring use of instrument fiducial markers disposed on the surgical instrument.

6. The apparatus according to claim 1, further comprising the imaging system, wherein the imaging system comprises one or more infrared cameras that are configured to acquire images of the veins within the portion of the patient's body.

7. The apparatus according to claim 1, further comprising the imaging system, wherein the imaging system comprises one or more depth cameras.

8. The apparatus according to claim 1, wherein the computer processor is configured to coregister the portion of the patient's body to the common coordinate system with the portion of the patient's body as it appears within the preoperative imaging data, by performing surface-matching registration between a surface of the portion of the patient's body as it appears within the preoperative imaging data and a current shape of the surface of portion of the patient's body.

9. The apparatus according to claim 8, wherein the computer processor is further configured to coregister the portion of the patient's body to the common coordinate system with the portion of the patient's body as it appears within the preoperative imaging data by modelling changes between the shapes of internal portions of the portion of the patient's body as the internal portions of the portion of the patient's body appear in preoperative imaging data and current shapes of internal portions of the portion of the patient's body appear, based upon the surface-matching registration and tissue that is present within the internal portions of the portion of the patient's body.

10. The apparatus according to claim 8, wherein the computer processor is further configured to coregister the portion of the patient's body to the common coordinate system with the portion of the patient's body as it appears within the preoperative imaging data by determining that the portion of the patient's body has been cut, and modifying the preoperative imaging data to create an accurate representation of the cut organ.

11. The apparatus according to claim 1, further comprising a light source, wherein the computer processor is configured to perform 3D reconstruction of the portion of the patient's body based on at least some of the intraoperative images by:
driving the light source to direct light toward the portion of the patient's body; and
detecting light that is reflected from the portion of the patient's body within the intraoperative images.

12. The apparatus according to claim 1, wherein the computer processor is configured to receive preoperative planning that is performed with respect to the preoperative imaging data of the portion of the patient's body and the computer processor is configured to drive the output device to display a current location of the surgical instrument with respect to the preoperative planning, based upon the coregistering.

13. The apparatus according to claim 1, further comprising the imaging system, wherein the imaging system comprises a hyperspectral camera, and the computer processor is configured to coregister the portion of the patient's body to a common coordinate system with the portion of the patient's body as it appears within the preoperative imaging data using imaging data acquired using the hyperspectral camera.

14. The apparatus according to claim 1, wherein the computer processor is configured for use with a surgical navigation system that is configured to coregister anatomy of the patient with the preoperative imaging data of the portion of the patient's body such that the patient's anatomy and the preoperative imaging data are registered with each other within a navigation system common coordinate system, wherein the computer processor is configured to coregister images acquired by the imaging system within the navigation system common coordinate system, and wherein in response to detecting that the portion of the patient's body has undergone a change in shape since the preoperative imaging data were acquired, the computer processor is configured to update a shape of the preoperative imaging data and registration of the preoperative imaging data within the navigation system common coordinate system to reflect the change in shape that the portion of the patient's body has undergone.

15. The apparatus according to claim 1, wherein the computer processor is configured to coregister the portion of the patient's body to a common coordinate system with the portion of the patient's body as it appears within the preoperative imaging data, subsequent to the portion of the patient's body having undergone movement, deformation and/or resection since the preoperative imaging data were acquired.

16. The apparatus according to claim 1, wherein the computer processor is configured to coregister the portion of the patient's body to a common coordinate system with the portion of the patient's body as it appears within the preoperative imaging data, while the portion of the patient's body undergoes intraprocedural movement, deformation and/or resection.

17. A method for use during a surgical procedure that is performed on a portion of a body of a patient using a surgical instrument, the apparatus comprising:
acquiring preoperative imaging data of the portion of the patient's body; and
during the surgical procedure:
acquiring intraoperative images of the portion of the patient's body and the surgical instrument; and
using at least one computer processor:
identifying the portion of the patient's body within the intraoperative images;
segmenting the portion of the patient's body within the intraoperative images;
performing 3D reconstruction of the portion of the patient's body based on at least some of the intraoperative images;
coregistering the portion of the patient's body to a common coordinate system with the portion of the patient's body as it appears within the preoperative imaging data using a non-rigid coregistration algorithm; and
displaying an output upon an output device, based upon the coregistering.

18. The apparatus according to claim 17, wherein displaying the output upon the output device comprises display a current location of the surgical instrument with respect to the preoperative imaging data on the output device, based upon the coregistering.

19. An apparatus for use during a surgical procedure that is performed on a portion of a body of a patient using a surgical instrument, an imaging system, an output device, fiducial markers placed upon the patient's body, and a surgical navigation system that is configured to coregister anatomy of the patient with the preoperative imaging data of the portion of the patient's body such that the patient's anatomy and the preoperative imaging data are registered with each other within a navigation system common coordinate system, by identifying the fiducial markers within images of the patient's body, the apparatus comprising:
at least one computer processor configured:
to receive preoperative imaging data of the portion of the patient's body and the surgical instrument;
segment the preoperative imaging data of the portion of the subject's body into substructures; and
during the surgical procedure:
to coregister the segmented substructure to the patient's body, such that the patient's body and the segmented substructures within the preoperative imaging data are registered within a navigation system common coordinate system;
to coregister images acquired by the imaging system within the navigation system common coordinate system;
to receive intraoperative images of the portion of the patient's body from the imaging system;
to identify the portion of the patient's body within the intraoperative images;
to segment the portion of the patient's body within the intraoperative images;
to perform 3D reconstruction of the portion of the patient's body based on at least some of the intraoperative images;
to coregister the portion of the patient's body to the preoperative imaging data within the navigation system common coordinate system, the coregistering comprising updating the preoperative imaging data by moving at least one of the segmented substructures relative to others of the segmented substructures; and
drive the output device to display an output, based upon the coregistering.

20. The apparatus according to claim 19, wherein the computer processor is configured to coregister the portion of the patient's body to the preoperative imaging data within the navigation system common coordinate system at least partially by deforming the portion of the patient's body within the preoperative imaging data, using a non-rigid coregistration algorithm.
